(19) United States
(12) Patent Application Publication
(10) Pub. No.: US 2011/0263013 A1
(43) Pub. Date: Oct. 27, 2011

(54) Compositions And Methods For Growing Embryonic Stem Cells

(76) Inventors: Michael J. Shamblott, Baltimore, MD (US); Michael Cohen, West Orange, NJ (US)

(21) Appl. No.: 12/996,557

(22) PCT Filed: Jun. 5, 2009
(86) PCT No.: PCT/US09/03436

§ 371 Date: Jul. 13, 2011

Related U.S. Application Data
(60) Provisional application No. 61/129,141, filed on Jun. 6, 2008.

(51) Int. Cl. C12N 5/0735; C12N 5/0789; C07K 14/47

(52) U.S. Cl. 435/347; 435/366; 530/350

(57) ABSTRACT

Methods for deriving and cultivating human embryonic stem (ES) cells and maintaining their pluripotency in culture is provided by utilizing human umbilical cord blood derived stem cells or secreted proteins obtained from the culture medium of human umbilical cord blood derived stem cells.

### BACKGROUND OF THE INVENTION

Human embryonic stem (HES) cells are pluripotent cells that have the ability to differentiate into almost all adult cell types therefore hold great promise for regenerative medicine. Embryonic stem cells are derived from the inner cell mass of preimplantation embryos. Embryonic stem cells are pluripotent and are capable of differentiating into cells derived from all three embryonic germ layers. The traditional method used to derive mouse and human embryonic stem cells involves the use of support cells termed feeder cells or layers. However, to maintain the HES cells in an undifferentiated state requires the use of mouse embryonic fibroblast feeder layer (Thompson J A et al., 1998). These support cells provide a poorly understood set of signals that promote the conversion from blastocyst inner cell mass (ICM) cells to proliferating embryonic stem cells. Most commonly, primary cultures of mouse embryo fibroblasts are used as support cells for both mouse and HES cultures. The requirement for support cells is not lost following derivation, and HES cell cultures are most commonly maintained on feeder layers until differentiation is desired. Since the signals supplied by support cells are not understood, it has been difficult to find substitute cell types or to remove cells altogether. For research purposes, support cells provide a source of experimental variability and cellular contamination to ES cultures but are not disabling in their impact.

Because the use of mouse feeder cells is associated with contamination risks such as pathogen transmission and viral infection (Richards M et al., 2002), HES cells derived and cultured on mouse feeder cells are not suitable for clinical application. Much effort has been put into the development and refinement of xeno-free culture system. Several feeder system derived from human origin have been developed including human fetal skin fibroblasts, adult fibroblasts (Richards et al, 2002), foreskin fibroblasts (Amit et al, 2003) and human embryonic fibroblasts from differentiated HES (Stojkovic P et al., 2005). Because the fetal tissues were derived from human abortuses, fetal derived feeder cells will not be easily accepted. Also, because current existing HES cells are all derived from mouse feeder layer, feeder cells from them will be considered as not biologically safe as well.

The ideal feeder cell should meet the following requirements: 1) support the undifferentiated growth, pluripotent and genetically normal state of HES in vitro; 2) plentiful supply of starting material; 3) realistic possibility of allogeneic matching to patient and the possibility of autologous matching; 4) easy to derive and culture in vitro; 5) capability of long term proliferation; 6) compatible with eventual clinical application of HES-derived cells.

Notwithstanding the recognition of the need for feeder cells meeting the above criteria, eliminating the need for feeder cells is a further desirable goal. Even human feeder layers potentially contaminate HES cells with allogeneic proteins or living cells, and the potential for contamination by infectious agents still exists. Similar undesirable properties exist when non-human feeder cells are used. Eliminating feeder cells has not been successful; when cultured in a standard culture environment in the absence of mouse embryonic fibroblasts as feeder cells, HES cells rapidly differentiate or fail to survive. Attempts have been made to replace the feeder or support cells using cell-free components or at least avoid non-human components or cells. While some replacements have shown short-term promising results, such attempts have proven insufficient to support robust, continued propagation. For example, WO/9920741 describes the growth of embryonic stem cells in a nutrient serum effective to support the growth of primate-derived primordial stem cells and a substrate of feeder cells or an extracellular matrix component derived from feeder cells. The medium further includes non-essential amino acids, an anti-oxidant, and growth factors that are either nucleosides or a pyruvate salt. U.S. Pat. No. 6,642,048 reports growth of embryonic stem cells in feeder-free culture, using conditioned medium from such cells. U.S. Pat. No. 6,800,480 describes a cell culture medium for growing primate-derived primordial stem cells comprising a low osmotic pressure, low endotoxin basic medium comprising a nutrient serum and an extracellular matrix derived from the feeder cells. The medium further includes non-essential amino acids, an anti-oxidant (for example, beta-mercaptoethanol), and, optionally, nucleosides and a pyruvate salt. Need exists for better medium that supports the long-term propagation of embryonic stem cells in a pluripotent state.

### SUMMARY OF THE INVENTION

In one embodiment, methods for cultivating human embryonic stem (HES) cells and maintaining the pluripotency thereof are provided by growing the HES cells in a culture medium comprising a feeder layer of umbilical cord blood derived stem cells, medium comprising secreted proteins from umbilical cord blood derived stem cells, or the combination thereof. In one embodiment, the umbilical cord blood derived stem cells are adherent, CD45^{neg}, HLA class II^{neg} stem cells. In another embodiment, the adherent, CD45^{neg}, HLA class II^{neg} stem cells are CD34^{neg}, CD106^{neg}, CD44^{pos} and CD90^{pos}. In another embodiment, the cells are CD31, CD34, CD50, CD106 negative, and positive for CD44, CD71, CD90. In another embodiment, the umbilical cord blood derived stem cells are fibroblast-like. In a further embodiment, the feeder layer of umbilical cord blood derived stem cells is treated to halt cell division. In another embodiment, a substrate is provided such as but not limited to collagen I, collagen IV, fibronectin, superfibronectin, laminin, heparan sulfate proteoglycan, entactin, or any combination thereof. In another embodiment, the substrate is an extracellular matrix, such as may be obtained from human embryonic germ cell derivatives, human umbilical cord blood stem cells, human mesenchymal stem cells, or human fibroblasts.

In another embodiment, a composition is provided for cultivating human embryonic stem (ES) cells and maintaining the pluripotency thereof comprising a feeder layer of human umbilical cord blood derived stem cells, secreted proteins from human umbilical cord blood derived stem cells, or the combination thereof. In one embodiment, the umbilical cord blood derived stem cells are adherent, CD45^{neg}, HLA class II^{neg} stem cells. In another embodiment, the adherent, CD45^{neg}, HLA class II^{neg} stem cells are CD34^{neg}, CD106^{neg}, CD44^{pos} and CD90^{pos}. In another embodiment, the cells are CD31, CD34, LD50, CD106 negative, and positive for CD44, CD71, CD90. In another embodiment, the umbilical cord blood derived stem cells are fibroblast-like. In another embodiment, the umbilical cord blood derived stem cells are treated to halt cell division. In another embodiment, the composition further comprises a substrate such as but not limited to collagen I, collagen IV, fibronectin, superfibronectin, laminin, heparan sulfate proteoglycan, entactin, or any combination thereof. In another embodiment, the substrate is extracellular matrix, such as but not limited to that obtained from human embryonic germ cell derivatives, human mesenchymal stem cells, human umbilical cord blood derived stem cells or human fibroblasts.

In another embodiment, a kit is provided for cultivating human embryonic stem (ES) cells and maintaining the pluripotency thereof, the kit comprising a first container of secreted proteins from human umbilical cord blood derived stem cells, a second container of substrate, and instructions for the use thereof. In one embodiment, the human umbilical cord blood derived stem cells are adherent, CD45^{neg}, HLA class II^{neg} stem cells. In another embodiment, the adherent, CD45^{neg}, HLA class II^{neg} stem cells are CD34^{neg}, CD106^{neg}, CD44^{pos} and CD90^{pos}. In another embodiment, the cells are CD31, CD34, CD50, CD106 negative, and positive for CD44, CD71, CD90. In another embodiment, the umbilical cord blood derived stem cells are fibroblast-like. In another embodiment, the substrate is by way of non-limiting example collagen I, collagen IV, fibronectin, superfibronectin, laminin, heparan sulfate proteoglycan, entactin, or any combination thereof. In another embodiment, the substrate is extracellular matrix, such as but not limited to extracellular matrix is obtained from human embryonic germ cell derivatives, human mesenchymal stem cells, human umbilical cord blood stem cells or human fibroblasts.

In another embodiment, a composition is provided comprising pluripotent human embryonic stem (ES) cells and secreted proteins from human umbilical cord blood derived stem cells, in combination with a substrate. In one embodiment, the umbilical cord blood derived stem cells are adherent, CD45^{neg}, HLA class II^{neg} stem cells. In another embodiment, the adherent, CD45^{neg}, HLA class II^{neg} stem cells are CD34^{neg}, CD106^{neg}, CD44^{pos} and CD90^{pos}. In another embodiment, the cells are CD31, CD34, CD50, CD106 negative, and positive for CD44, CD71, CD90. In another embodiment, the umbilical cord blood derived stem cells are fibroblast-like. In another embodiment, the substrate is by way of non-limiting example, collagen I, collagen IV, fibronectin, superfibronectin, laminin, heparan sulfate proteoglycan, entactin, or any combination thereof. In another embodiment, the substrate is extracellular matrix, such as but not limited to extracellular matrix is obtained from human embryonic germ cell derivatives, human mesenchymal stem cells, human umbilical cord blood stem cells or human fibroblasts

In another embodiment, a composition is provided comprising pluripotent human embryonic stem (ES) cells and human umbilical cord blood derived stem cells. In one embodiment, the umbilical cord blood derived stem cells are adherent, CD45^{neg}, HLA class II^{neg} stem cells. In another embodiment, the adherent, CD45^{neg}, HLA class II^{neg} stem cells are CD34^{neg}, CD106^{neg}, CD44^{pos} and CD90^{pos}. In another embodiment, the cells are CD31, CD34, CD50, CD106 negative, and positive for CD44, CD71, CD90. In another embodiment, the umbilical cord blood derived stem cells are fibroblast-like. In another embodiment, the human umbilical cord blood derived stem cells are treated to halt cell division. In another embodiment, the composition further comprises a substrate, such as but not limited to collagen I, collagen IV, fibronectin, superfibronectin, laminin, heparan sulfate proteoglycan, entactin, or any combination thereof. In another embodiment, the substrate is extracellular matrix, such as but not limited to extracellular matrix obtained from human embryonic germ cell derivatives, human mesenchymal stem cells, human umbilical cord blood stem cells or human fibroblasts. In another embodiment, cultured pluripotent human embryonic stem (ES) cells are provided that are obtained by the process of 1) providing a culture medium comprising a composition as described above, 2) introducing human embryonic stem cells thereto; and 3) growing the human embryonic stem cells therein to produce cultured pluripotent human embryonic stem cells.

In another embodiment, a method for obtaining a pluripotent human embryonic cell line is provided comprising the steps of 1) isolating cells from the inner cell mass of a pre-implantation embryo, 2) introducing the cells of (1) into a culture medium comprising a composition described above, 3) growing the human embryonic stem cells over several passages in the culture medium, thereby obtaining a human embryonic cell line derived from the pre-implantation embryo.

### BRIEF DESCRIPTION OF THE DRAWINGS

The invention will be better understood from a reading of the following detailed description taken in conjunction with the drawings in which like reference designators are used to designate like elements, and in which:

FIG. 1A-F show the immunophenotype of umbilical cord blood stem cells (passage 5) derived from umbilical cord blood mononuclear cells. A, CD31; B, CD106; C, CD50; D, CD44; E, CD 90 and F, CD71. The isotype negative control staining shown in dark gray, specific staining shown in light gray. Percentage of cells staining positive shown under the isotype negative control gate;

FIG. 2 A-B show the morphology of HESs grown on umbilical cord blood derived stem cells feeder layer for 10 passages. A, H1 cells; B, H9 cells. Size bar is 100 microns;

FIG. 3 A-H show the characteristics of HES lines H1 (A,C,E,G) and H9 (B,D,F,H) grown on umbilical cord blood derived stem cell feeder layers (passage 20). Cells are stained with antibodies recognizing: Oct-4(A,B); SSEA-4, (C,D); Tra-1-60, (E,F) and Tra-1-81, (G,H). Cell nuclei stained with DAPI. Size bars are 50 microns;

FIG. 4 A-B show a karyotype analyses of HES lines H1 and H9 grown on umbilical cord blood derived stem cell feeder cells. A, H1 (passage 21); B, 119 (passage 20);

FIG. 5 A-B show embryoid bodies formed from HES cell lines A, H1 and B, H9. Size bars are 200 microns; and

FIG. 6 A-D show a histological analysis of teratomas formed from grafted colonies of hESC line H9 line (passage 20) grown on umbilical cord blood derived stem cell feeder cells. A, neural rosettes (ectoderm); B, mucus-producing epithelium (endoderm); C, cartilage (mesoderm); D, bone (mesoderm).

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

While the therapeutic and other applications of embryonic stem (ES) cells are projected to have a major impact on the future of health care and the treatment of a large number of diseases, methods for deriving ES cells from the embryo and maintaining the pluripotency of thus-derived ES cells in a medium readily compatible with human administration has hindered progress in this field. While ES cell can be derived from blastocyst inner mass cells and maintained in a pluripotent state using mouse feeder cell layers and conditioned medium from mouse feeder cells, use of any mouse products at any point during the preparation of ES cells for human therapy has adverse regulatory implications. The inventors herein, in an effort to identify substitutes for mouse embryo fibroblasts in deriving and maintaining ES cells, discovered that stem cells obtained from umbilical cord blood, and in one non-limiting embodiment, cells with a CD45^{neg}, HLA class II^{neg} phenotype, provide the necessary feeder layer to support the derivation and propagation of ES cells. In another embodiment the cells have a CD34^{neg}, CD106^{neg} and CD44^{pos} and CD90^{pos} phenotype. In another embodiment, the cells are CD31, CD34, LD50, CD106 negative, and positive for CD44, CD71, CD90. In another embodiment, the umbilical cord blood derived stem cells are spindle-like or fibroblast-like. Furthermore, the inventors herein also found secreted proteins produced by the aforementioned human umbilical cord blood derived stem cells comprise the necessary components to permit both the derivation and propagation of ES cells in the absence of any mouse-derived materials (both cells and secreted proteins including extracellular matrix). Thus, human umbilical cord blood derived stem cells can be used as feeder layers for ES cells, used to produce conditioned medium for ES cells, used to produce extracellular matrix for ES cells, or any combination thereof.

As will be noted in the Examples below, two HES cell lines (H1 and H9) were successfully cultured on human umbilical cord blood derived fibroblast-like cells as described herein. These cells permit the long term continuous growth of undifferentiated and pluripotent HES cells. The cultured HES cells had normal karyotypes, expressed Oct-4, SSEA-4, TRA-1-60 and TRA-1-81, formed cystic embryonic body in vitro and teratomas in vivo after injected into immunodeficient mice. The wide availability of clinical-grade human umbilical cord blood makes it a promising source of support cells for the growth of HES cells for use in cell therapies.

Moreover, as will also be shown in the examples, HES cells grown in accordance with the teachings herein maintain pluripotency. Using either human umbilical cord blood derived stem cells as a feeder layer, or using their secreted proteins (conditioned medium) therefrom in the growth medium, ES cells did not differentiate at the colony edges or center, and remained undifferentiated. In another embodiment, ES cells grown as described herein readily disaggregated from the feeder layers after trypsin treatment, affording an easier method for recovering cells from culture than when other feeder layers are used.

Other substrates that can be used in combination with feeder layers or conditioned medium include, by way of non-limiting example, collagen I, collagen IV, fibronectin, superfibronectin, laminin, heparan sulfate proteoglycan, entactin, or any combination thereof. Typically, the collagen I is human type I collagen. Typically a substrate of human origin is used in order to avoid the presence of non-human components in ES cultures, but for purposes other than human therapeutic uses, non-human components may be present. In another embodiment, the substrate comprises any synthetic or biosynthetic cell adhesion molecule or a mixture thereof.

The aforementioned substrates such as collagen I and fibronectin or superfibronectin can be purchased as purified proteins or proteoglycans from any number of suppliers (such as Sigma Chemical Company, Innovative Research or Research Diagnostics Inc.) or prepared and purified in the laboratory. Fibronectin is an extracellular matrix protein that is important in development, wound healing and tumorigenesis. In the blood it is dimeric, but in tissues forms disulphide crosslinked fibrils. Superfibronectin is derived using a fragment from the first type-III repeat of fibronectin which binds to fibronectin and induces spontaneous disulphide crosslinking of the molecule into multimers of high relative molecular mass which resemble matrix fibrils. Treatment of fibronectin with this inducing fragment converts fibronectin into a form that has greatly enhanced adhesive properties (hence the term superfibronectin) and which suppresses cell migration [14].

In addition to the aforementioned substrates, other synthetic or biosynthetic adhesion molecules can be used, including fragments and peptides from the aforementioned proteins that support growth of ES cells.

In another embodiment, the aforementioned method further comprises the use of an extracellular matrix. Extracellular matrix may be obtained from normal cells or immortalized cell lines. Non-limiting examples include extracellular matrix from human embryonic germ (EG) cell derivatives, such as from human embryoid body-derived cells. Non-limiting examples of such cells include LVEC cells or SDEC cells. In another embodiment, the extracellular matrix is EHS mouse sarcoma basement membrane or human extracellular matrix. As noted above, typically a human extracellular matrix is used in order to avoid the presence of non-human components in ES cultures, but for purposes other than human therapeutic uses, non-human components may be present. In addition to the examples above human extracellular matrix can be obtained from any human cell type.

In addition to methods for growing ES cells and maintaining their pluripotency, compositions are provided therefor. Such compositions can comprise a feeder layer of human umbilical cord blood derived stem cells, optionally in combination with secreted proteins from such cells, optionally in combination with a substrate. The human umbilical cord blood derived stem cells can be cells with a CD45^{neg}, HLA class II^{neg} phenotype, or in a further embodiment the cells have a CD34^{neg}, CD106^{neg} and CD44^{pos} and CD90^{pos} phenotype. In another embodiment, the cells are CD31, CD34, CD50, CD106 negative, and positive for CD44, CD71, CD90. In another embodiment, the umbilical cord blood derived stem cells are fibroblast-like. In another embodiment, the feeder layer cells are treated to halt cell division, such as by exposure to gamma radiation or exposure to mitomycin. Conditioned medium can be obtained from the growth of the aforementioned cells in culture medium. The substrate optionally present in the aforementioned composition can be collagen 1, collagen IV, fibronectin, superfibronectin, laminin, heparan sulfate proteoglycan, entactin, or any combination thereof. Typically, the collagen I is human type I collagen. Other synthetic or biosynthetic adhesion molecules may also be used. Typically a substrate of human origin is used in order to avoid the presence of non-human components in ES cultures, but for purposes other than human therapeutic uses, non-human components may be present. In another embodiment, the substrate is an extracellular matrix, such as that obtained from human embryonic germ (EG) cell derivatives, typically human embryoid body-derived cells. Non-limiting examples include LVEC cells or SDEC cells. In another embodiment, the extracellular matrix is EHS mouse sarcoma basement membrane or human extracellular matrix.

In yet another embodiment, a kit is provided for cultivating human embryonic stem cells and maintaining the pluripotency thereof, the kit comprising a first container secreted proteins from human umbilical cord blood derived stem cells, a second container of substrate, and instructions for the use thereof. In one embodiment, the human umbilical cord blood derived stem cells are cells with a CD45^{neg}, HLA class II^{neg} phenotype. In another embodiment the cells have a CD34^{neg}, CD106^{neg} and CD44^{pos} and CD90^{pos} phenotype. In another embodiment, the cells are CD31, CD34, CD50, CD106 negative, and positive for CD44, CD71, CD90. In another embodiment, the umbilical cord blood derived stem cells are fibroblast-like. The substrate can be collagen I, collagen IV, fibronectin, superfibronectin, laminin, heparan sulfate proteoglycan, entactin, or any combination thereof. Typically the collagen I is human type I collagen. Other synthetic or biosynthetic adhesion molecules or mixtures may also be used. Typically a substrate of human origin is used in order to avoid the presence of non-human components in ES cultures, but for purposes other than human therapeutic uses, non-human components may be present. In another embodiment the substrate can be an extracellular matrix, such as that obtained from human embryonic germ (EG) cell derivatives, for example, human embryoid body-derived cells such as but not limited to LVEC cells or SDEC cells. The extracellular matrix can be EHS mouse sarcoma basement membrane or human extracellular matrix.

Another embodiment of the invention is a composition comprising pluripotent human embryonic stem (ES) cells and secreted proteins from human umbilical cord blood derived stem cells. In one embodiment, the human umbilical cord blood derived stem cells are cells with a CD45^{neg}, HLA class II^{neg} phenotype. In another embodiment the cells have a CD34^{neg}, CD106^{neg} and CD44^{pos} and CD90^{pos} phenotype. In another embodiment, the cells are CD31, CD34, CD50, CD106 negative, and positive for CD44, CD71, CD90. In another embodiment, the umbilical cord blood derived stem cells are fibroblast-like. The composition can further comprise a substrate, such as but not limited to collagen I, collagen IV, fibronectin, superfibronectin, laminin, heparan sulfate proteoglycan, entactin, or any combination thereof. Typically the collagen I is human type 1 collagen. In another embodiment, the substrate is a synthetic or biosynthetic adhesion molecule or a mixture thereof. Typically a substrate of human origin is used in order to avoid the presence of non-human components in ES cultures, but for purposes other than human therapeutic uses, non-human components may be present. In another embodiment, the composition can include an extracellular matrix. The extracellular matrix can be obtained from human embryonic germ (EG) cell derivatives, typically human embryoid body-derived cells. Non-limiting examples include LVEC cells or SDEC cells. The extracellular matrix can be EHS mouse sarcoma basement membrane or human extracellular matrix.

Another embodiment of the invention is a composition comprising pluripotent human embryonic stem (ES) cells and human umbilical cord blood derived stem cells. In one embodiment, the human umbilical cord blood derived stem cells are cells with a CD45^{neg}, HLA class II^{neg} phenotype. In another embodiment the cells have a CD34^{neg}, CD106^{neg} and CD44^{pos} and CD90^{pos} phenotype. In another embodiment, the cells are CD31, CD34, CD50, CD106 negative, and positive for CD44, CD71, CD90. In another embodiment, the umbilical cord blood derived stem cells are fibroblast-like. The composition can further comprise a substrate, such as but not limited to collagen I, collagen IV, fibronectin, superfibronectin, laminin, heparan sulfate proteoglycan, entactin, or any combination thereof. Typically the collagen I is human type 1 collagen. In another embodiment, the substrate is a synthetic or biosynthetic adhesion molecule or a mixture thereof. Typically a substrate of human origin is used in order to avoid the presence of non-human components in ES cultures, but for purposes other than human therapeutic uses, non-human components may be present. In another embodiment, the composition can include an extracellular matrix. The extracellular matrix can be obtained from human embryonic germ (EG) cell derivatives, typically human embryoid body-derived cells. Non-limiting examples include LVEC cells or SDEC cells. The extracellular matrix can be EHS mouse sarcoma basement membrane or human extracellular matrix.

In another embodiment of the invention, cultured pluripotent human embryonic stem (ES) cells can be obtained by the process of 1) providing a culture medium comprising secreted proteins from human umbilical cord blood derived stem cells, together with a substrate, 2) introducing human embryonic stem (ES) cells thereto; and 3) growing the human embryonic stem (ES) cells therein to produce cultured pluripotent human embryonic stem cells. In one embodiment, the human umbilical cord blood derived stem cells are cells with a CD45^{neg}, HLA class II^{neg} phenotype. In another embodiment the cells have a CD34^{neg}, CD106^{neg} and CD44^{pos} and CD90^{pos} phenotype. In another embodiment, the cells are CD31, CD34, CD50, CD106 negative, and positive for CD44, CD71, CD90. In another embodiment, the umbilical cord blood derived stem cells are fibroblast-like. In one embodiment, human umbilical cord blood derived stem cells are included in the cultures. The substrate can be collagen I, collagen IV, fibronectin, superfibronectin, laminin, heparan sulfate proteoglycan, entactin, or any combination thereof. Typically, the collagen I is human type 1 collagen. In another embodiment, the substrate is a synthetic or biosynthetic adhesion molecule or a mixture thereof. Typically a substrate of human origin is used in order to avoid the presence of non-human components in ES cultures, but for purposes other than human therapeutic uses, non-human components may be present. In another embodiment, the substrate is extracellular matrix, for example, extracellular matrix is obtained from embryonic germ (EG) cell derivatives, typically human embryoid body-derived cells, such as LVEC cells or SDEC cells.

In another embodiment of the invention, cultured pluripotent human embryonic stem (ES) cells can be obtained by the process of 1) providing a culture medium comprising human umbilical cord blood derived stem cells, together with a substrate, 2) introducing human embryonic stem (ES) cells thereto; and 3) growing the human embryonic stem (ES) cells therein to produce cultured pluripotent human embryonic stem cells. In one embodiment, the human umbilical cord blood derived stem cells are cells with a CD45^{neg}, HLA class II^{neg} phenotype. In another embodiment the cells have a CD34^{neg}, CD 106^{neg} and CD44^{pos} and CD90^{pos} phenotype. In another embodiment, the cells are CD31, CD34, CD50, CD106 negative, and positive for CD44, CD71, CD90. In another embodiment, the umbilical cord blood derived stem cells are fibroblast-like. In another embodiment, the human umbilical cord blood derived stem cells are treated to halt cell division, such as by exposure to gamma radiation or exposure to mitomycin.

In another embodiment, secreted proteins from human umbilical cord blood derived stem calls are included in the medium. The substrate can be collagen I, collagen IV, fibronectin, superfibronectin, laminin, heparan sulfate proteoglycan, entactin, or any combination thereof. Typically, the collagen I is human type 1 collagen. In another embodiment, the substrate is a synthetic or biosynthetic adhesion molecule or a mixture thereof. Typically a substrate of human origin is used in order to avoid the presence of non-human components in ES cultures, but for purposes other than human therapeutic uses, non-human components may be present. In another embodiment, the substrate is extracellular matrix, for example, extracellular matrix is obtained from embryonic germ (EG) cell derivatives, typically human embryoid body-derived cells, such as LVEC cells or SDEC cells.

In yet another embodiment of the invention, a method is provided for obtaining a pluripotent human embryonic cell line comprising the steps of 1) isolating human cells from the inner cell mass of a pre-implantation embryo, 2) introducing the cells of (1) into a culture medium comprising a composition as described above; and 3) growing the human embryonic stem cells derived thereby over several passages in the culture medium, thereby obtaining a human embryonic cell line derived from the pre-implantation embryo. Such compositions can comprise a feeder layer of human umbilical cord blood derived stem cells, optionally in combination with secreted proteins from such cells, optionally in combination with a substrate. The human umbilical cord blood derived stem cells can be cells with a CD45^{neg}, HLA class II^{neg} phenotype, or in a further embodiment the cells have a CD34^{neg}, CD106^{neg} and CD44^{pos} and CD90^{pos} phenotype. In another embodiment, the cells are CD31, CD34, CD50, CD106 negative, and positive for CD44, CD71, CD90. In another embodiment, the umbilical cord blood derived stem cells are fibroblast-like. In another embodiment, the feeder layer cells are treated to halt cell division, such as by exposure to gamma radiation or exposure to mitomycin. Conditioned medium can be obtained from the growth of the aforementioned cells in culture medium and used together with or in place of the human umbilical cord blood derived stem cells. The substrate optionally present in the aforementioned composition can be collagen I, collagen IV, fibronectin, superfibronectin, laminin, heparan sulfate proteoglycan, entactin, or any combination thereof. Typically, the collagen I is human type I collagen. Other synthetic or biosynthetic adhesion molecules may also be used. Typically a substrate of human origin is used in order to avoid the presence of non-human components in ES cultures, but for purposes other than human therapeutic uses, non-human components may be present. In another embodiment, the substrate is an extracellular matrix, such as that obtained from human embryonic germ (EG) cell derivatives, typically human embryoid body-derived cells. Non-limiting examples include LVEC cells or SDEC cells. In another embodiment, the extracellular matrix is EHS mouse sarcoma basement membrane or human extracellular matrix.

The following sections provide descriptions of each of the components of the present invention. They are intended to be exemplary only and non-limiting, and one of ordinary skill will recognize alternative means for achieving the same result within the spirit of the invention.

Substrates. The aforementioned substrates collagen I, collagen IV, fibronectin, superfibronectin, laminin, heparan sulfate proteoglycan, entactin, singly or in any combination, are used in an embodiment wherein the substrate is a defined protein or combination of proteins. These proteins are readily available commercially or can be prepared in the laboratory following guidance in the art. Typically human proteins are used in the practice of the invention but this is not so limiting if human therapeutic use is not contemplated.

Extracellular Matrix. Extracellular matrix can be purchased or prepared from cells in accordance with teachings in the art. One example of a mouse extracellular matrix favored in work prior to the invention described herein is EHS mouse sarcoma basement membrane, manufactured by BD Biosciences (San Jose, Calif.) and sold under the name MATRIGEL. A human extracellular matrix is also commercially available from BD Biosciences. Typically, the invention is carried out using type I collagen. In another embodiment, extracellular matrix produced by embryonic germ (EG) cell derivatives, such as SDEC and LVEC cells (Shamblott et al., 2001) as described herein, can be used.

In another embodiment, the substrate comprises any synthetic or biosynthetic cell adhesion molecule. Among the substrates described above, fragments and peptides thereof capable of supporting growth of ES cells are further embodiments of the invention. In one embodiment, a peptide comprising the tripeptide RGD is useful as a substrate for the purposes herein described.

Extracellular matrix from human embryonic germ cell derivatives. In the practice of the invention, human embryonic germ (EG) cell derivatives may be used as a source of the extracellular matrix that supports derivation and growth of ES cells. EG cells can be generated and cultured essentially as described in U.S. Pat. No. 6,090,622. The starting material for isolating cultured embryonic germ (EG) cells are tissues and organs comprising primordial germ cells (PGCs). For example, PGCs may be isolated over a period of about 3 to 13 weeks post-fertilization (e.g., about 9 weeks to about 11 weeks from the last menstrual period) from embryonic yolk sac, mesenteries, gonadal anlagen, or genital ridges from a human embryo or fetus. Alternatively, gonocytes of later testicular stages can also provide PGCs. In one embodiment, the PGCs are cultured on mitotically inactivated fibroblast cells (e.g., STO cells) under conditions effective to derive EGs. The resulting human EG cells resemble murine ES or EG cells in morphology and in biochemical histotype. The resulting human EG cells can be passaged and maintained for at least several months in culture.

As noted above and in the examples, below, use of umbilical cord blood derived stem cells as feeder cells or use of their secreted proteins as a growth medium affords additional benefits for the growth and harvesting of pluripotent ES cells, over other feeders cells or conditioned medium. In one embodiment, ES cells grown according to the teachings herein maintain pluripotency in culture and do not show differentiation at the edges or center of colonies. Thus, pluripotency is maintained. Furthermore, harvesting of cells by trypsinization affords ready disaggregation of colonies and easy removal from feeder layers of human embryonic stem cells. The various exemplary uses of ES cells described below benefit from the foregoing advantages provided by the teachings herein.

The development of an optimal feeder layer system of human origin for HES cell culture is critical for future clinical application. Because of the possible genetic instability of HES cells in the feeder-free conditions, a feeder layer system for HES cell maintenance in vitro may be preferred. The use of the umbilical cord blood derived stem cells for this purpose is fully embodied herein.

### Uses of ES Cells Derived or Propagated as Describe Herein.

Cell-Based Therapies: Transplantation of ES Cells. The invention also provides methods for growth of unmodified or genetically modified ES cells or their differentiated progeny for use in human transplantations in the fetus, newborns, infants, children, and/or adults. One example of this use is therapeutic supplementation of metabolic enzymes for the treatment of autosomal recessive disorders. For example, production of homogentisic acid oxidase by transplanted ES differentiated cells into the liver could be used in the treatment of alkaptonuria (for review of this disorder, see McKusick, Heritable Disorders of Connective Tissue. 4th ed., St. Louis, C. V. Mosby Co., 1972). Likewise, ornithine transcarbamylase expression could be augmented to treat the disease caused by its deficiency. In another example, glucose-6-phosphate dehydrogenase expression could be augmented in erythrocyte precursors or hematopoietic precursors to allow expression in red blood cells in order to treat G6PD deficiency (favism, acute hemolytic anemnia).

Treatments of some diseases require addition of a composition or the production of a circulating factor. One example is the production of alpha1-antitrypsin in plasma to treat a deficiency that causes lung destruction, especially in tobacco smokers. Other examples of providing circulating factors are the production of hormones, growth factors, blood proteins, and homeostatic regulators.

In another embodiment of the invention, differentiated ES cells obtained or grown as described herein are used to repair or supplement damaged or degenerating tissues or organs. This may require that the cells are first differentiated in vitro into lineage-restricted stem cells or terminally differentiated cells.

Before implantation or transplantation the ES cell obtained or grown as described herein can be genetically manipulated to reduce or remove cell-surface molecules responsible for transplantation rejection in order to generate universal donor cells. For example, the mouse Class I histocompatibility (MHC) genes can be disabled by targeted deletion or disruption of the beta-microglobulin gene (see, e.g., Zijlstra, Nature 342:435-438, 1989). This significantly improves renal function in mouse kidney allografts (see, e.g., Coffman, J. Immunol. 151:425-435, 1993) and allows indefinite survival of murine pancreatic islet allografts (see, e.g., Markmann, Transplantation 54:1085-1089, 1992). Deletion of the Class II MHC genes (see, e.g., Cosgrove, Cell 66:1051-1066, 1991) further improves the outcome of transplantation. The molecules TAP1 and Ii direct the intercellular trafficking of MHC class I and class II molecules, respectively (see, e.g., Toume, Proc. Natl. Acad. Sci. USA 93:1464-1469, 1996); removal of these two transporter molecules, or other MHC intracellular trafficking systems may also provide a means to reduce or eliminate transplantation rejection. As an alternative to a universal donor approach to histocompatibility, genetic manipulation could be used to generate “custom” MHC profiles to match individual needs.

In addition to manipulating MHC expression, for human transplantation, cells and tissues from ES cells and cell lines grown in accordance with the invention can also be manipulated to eliminate or reduce other cell-surface marker molecules that induce tissue/organ graft rejection. All such modifications that reduce or eliminate allogenic (e.g., organ graft) rejection when employing cells, cell lines (or any parts or derivatives thereof) derived from the cells of the present invention are embodied herein.

Tissue Engineering. The invention provides human cells and methods that can be used to produce or reconstruct a tissue or organ, including in vitro or vivo regeneration, and engineering of artificial organs or organoids. In one aspect, the ES cells grown in accordance with the invention are pre-cultured under conditions that promote generation of a desired differentiated, or restricted, cell lineage. The culture conditions can also be manipulated to generate a specific cell architecture, such as the three-dimensional cellular arrangements and relationships seen in specialized structures, such as neuromuscular junctions and neural synapses, or organs, such as livers, and the like. These conditions can include the use of bioreactor systems to influence the generation of the desired cell type. Bioreactor systems are commonly used in the art of tissue engineering to create artificial tissues and organs. Some bioreactor systems are designed to provide physiological stimuli similar to those found in the natural environments. Others are designed to provide a three-dimensional architecture to develop an organ culture. For example, the compositions (including bioreactors, scaffolds, culture devices, three-dimensional cell culture systems, and the like) and methods described in U.S. Pat. Nos. 6,143,293; 6,121,042; 6,110,487; 6,103,255; 6,080,581; 6,048,721; 6,022,743; 6,022,742; 6,008,049; 6,001,642; 5,989,913; 5,962,325; 5,858,721; 5,843,766; 5,792,603; 5,770,417; 5,763,279; 5,688,687; 5,612,188; 5,571,720; 5,770,417; 5,626,863; 5,523,228; 5,459,069; 5,449,617; 5,424,209; 5,416,022; 5,266,480; 5,223,428; 5,041,138; and 5,032,508; or variations thereof, can be used in conjunction with this invention.

As discussed above, production of cells, tissues and organs for transplantation may require combinations of genetic modifications, in vitro differentiation, and defined substrate utilization of the cells of the invention to generate the desired altered cell phenotype and, if a tissue or organ is to be generated, the necessary three-dimensional architecture required for functionality. For example, a replacement organ may require vasculature to deliver nutrients, remove waste products, and deliver products, as well as specific cell-cell contacts. A diverse cell population will be required to carry out these and other specialized functions, such as the capacity to repopulate by lineage-restricted stem cells.

Further examples of the use of the ES cells obtained or grown in accordance with the invention and their differentiated derivatives include generation of non-cellular structures such as bone or cartilage replacements.

Human ES cells obtained or grown in accordance with the invention can also be implanted into the central nervous system (CNS) for the treatment of disease or physical brain injury, such as ischemia or chemical injury; animal models can also be used to test the efficacy of this treatment, e.g., injection of compounds like 60HAD, or, fluid percussion injury can serve as a model for human brain injury. In these animal models, the efficacy of administration of stem cells of the invention is determined by the recovery of improvement of injury related deficits, e.g., motor or behavioral deficits. Human ES cells obtained in accordance with the invention can also be implanted into the central nervous system (CNS) for the treatment of amyotropic lateral sclerosis (ALS); animal models can also be used to test the efficacy of this treatment, e.g., the SODI mutant mouse model. Human ES cells of the invention can also be implanted into the central nervous system (CNS) for the treatment of Alzheimer's disease; one animal model that can be used to test the efficacy of this treatment is the mutant presenilin I mouse. Human ES cells can also be implanted into the central nervous system (CNS) for the treatment of Parkinson's disease, efficacy of this treatment can be assessed using, e.g., the MPTP mouse model.

Human ES cells grown in accordance with the invention can also be used to treat diseases of cardiac, skeletal or smooth muscles; cells can be directly injected into or near desired sites. The survival and differential of these cells can be determined by monitoring the expression of appropriate markers, e.g, human muscle-specific gene products (see, e.g., Klug, 1996, supra; Soonpaa, Science 264:98-101, 1994; Klug, Am. J. Physiol. 269:H1913-H1921, 1995; implanting fetal cardiomyocytes and mouse ES-derived cells), for exemplary protocols.

Human ES cells grown in accordance with the invention can also be used to treat diseases of the liver or pancreas. Cells can be directly injected into the hepatic duct or the associated vasculature. Similarly, cells could be delivered into the pancreas by direct implantation or by injection into the vasculature. Cells engraft into the liver or pancreatic parenchyma, taking on the functions normally associated with hepatocytes or pancreatic cells, respectively. As with other implantations, cell survival, differentiation and function can be monitored by, e.g., immunohistochemical staining, or PCR, of specific gene products.

Human ES cells of the invention can also be used to treat diseases, injuries or other conditions in or related to the eyes. Cells can be directly injected into the retina, optic nerve or other eye structure. In one aspect, cells differentiate into retinal epithelia, nerve cells or other related cell types. As with other engraftments, cell survival, differentiation and function can be monitored by, e.g., immunohistochemical staining, or PCR, of specific gene products.

Human ES cells of the invention can also be used to treat vascular diseases or other related conditions by repopulation of the vasculature with, e.g., vascular endothelium, vascular smooth muscle and other related cell types. For example, an injured vein or artery is treated by implantation of ES cells of the invention; these cells re-populate the appropriate injured sites in the vasculature. The cells can be implanted/injected into the general circulation, by local (“regional”) injection (e.g., into a specific organ) or by local injection, e.g., into a temporarily isolated region. In an alternative procedure, a reconstructed or a completely new vasculature can be constructed on a biomatrix or in an organotypic culture, as described herein.

Human ES cells of the invention can also be used to repopulate bone marrow, e.g., in situations where bone marrow has been ablated, e.g., by irradiation for the treatment of certain cancers. Protocols for these treatments can be optimized using animal models, e.g., in animals whose endogenous bone marrow has been ablated. EBD cells of the invention can be injected into the circulatory system or directly into the marrow space of such an animal (e.g., a rodent model). Injection of the human cells of the invention would allow for the re-population of bone marrow, as well as engraftment of a wide range of tissues and organs. If the animals are sublethally irradiated, the efficacy of the cells can be monitored by tracking animal survival, as without bone marrow re-population the animal will die. The hematopoietic fate of the injected cells also can be examined by determining the type and amount to human cell colonies in the spleen.

In another aspect, the human ES cells obtained or grown in accordance with the invention can be used in organotypic co-culture. This system offers the benefits of direct cell application and visualization found in in vitro methods with the complex and physiologically relevant milieu of an in vivo application. In one aspect, a section of tissue or an organ specimen is placed into a specialized culture environment that allows sufficient nutrient access and gas exchange to maintain cellular viability.

In using the human ES cells, or differentiated derivatives thereof, of the invention to construct artificial organs or organoids, bioengineered matrices or lattice structures can be populated by single or successive application of these human cells. The matrices can provide structural support and architectural cues for the repopulating cells.

Biosensors and Methods of Screening. ES cells or cell lines obtained or grown in accordance with the invention and cells, tissues, structures and organs derived from them can be used for toxicological, mutagenic, and/or teratogenic in vitro tests and as biosensors. Thus, the invention provides engineered cells, tissues and organs for screening methods to replace animal models and form novel human cell-based tests. These systems are useful as extreme environment biosensors. ES cells or cell lines and cells, tissues, structures and organs derived from them can be used to build physiological biosensors; for example, they can be incorporated in known system, as described, e.g., in U.S. Pat. Nos. 6,130,037; 6,129,896; and 6,127,129. These sensors can be implanted bio-electronic devices that function as in vivo monitors of metabolism and other biological functions, or as an interface between human and computer.

The invention also provides a method for identifying a compound that modulates an ES cell function in some way (e.g., modulates differentiation, cell proliferation, production of factors or other proteins, gene expression). The method includes: (a) incubating components comprising the compound and ES cell(s) grown under conditions described herein, sufficient to allow the components to interact; and (b) determining the effect of the compound on the ES cell(s) before and after incubating in the presence of the compound. Compounds that ES cell function include peptides, peptidomimetics, polypeptides, chemical compounds and biologic agents. Differentiation, gene expression, cell membrane permeability, proliferation and the like can be determined by methods commonly used in the art. The term “modulation” refers to inhibition, augmentation, or stimulation of a particular cell function.

ES Cells as Sources of Macromolecules. The ES cells and cell lines obtained or grown in accordance with the invention can also be used in the biosynthetic production of macromolecules. Non-limiting examples of products that could be produced are blood proteins, hormones, growth factors, cytokines, enzymes, receptors, binding proteins, signal transduction molecules, cell surface antigens, and structural molecules. Factors produced by undifferentiated, differentiating, or differentiated ES cells would closely simulate the subtle folding and secondary processing of native human factors produced in vivo. Biosynthetic production by ES cells and cell lines can also involve genetic manipulation followed by in vitro growth and/or differentiation. Biosynthetic products can be secreted into the growth media or produced intracellularly or contained within the cell membrane, and harvested after cell disruption. Genetic modification of the gene coding for the macromolecule to be biosynthetically produced can be used to alter its characteristics in order to supplement or enhance functionality. In this way, novel enhanced-property macromolecules can be created and pharmaceuticals, diagnostics, or antibodies, used in manufacturing or processing, can be produced. Pharmaceutical, therapeutic, processing, manufacturing or compositional proteins that may be produced in this manner include, e.g., blood proteins (clotting factors VIII and IX, complement factors or components, hemoglobins or other blood proteins and the like); hormones (insulin, growth hormone, thyroid hormone, gonadotrophins, PMSG trophic hormones, prolactin, oxytocin, dopamine, catecholamines and the like); growth factors (EGF, PDGF, NGF, IGF and the like); cytokines (interleukins, CSF, GMCSF, TNF, TGF.alpha., TGF.beta., and the like); enzymes (tissue plasminogen activator, streptokinase, cholesterol biosynthetic or degradative, digestive, steroidogenic, kinases, phosphodiesterases, methylases, de-methylases, dehydrogenases, cellulases, proteases, lipases, phospholipases, aromatase, cytochromes adenylate or guanylate cyclases and the like); hormone or other receptors (LDL, HDL, steroid, protein, peptide, lipid or prostaglandin and the like); binding proteins (steroid binding proteins, growth hormone or growth factor binding proteins and the like); immune system proteins (antibodies, SLA or MHC gene products); antigens (bacterial, parasitic, viral, allergens, and the like); translation or transcription factors, oncoproteins or proto-oncoproteins, milk proteins (caseins, lactalbumins, whey and the like); muscle proteins (myosin, tropomyosin, and the like).

Screens for Culture Media Factors. In another embodiment and use of the invention, ES cells grown in accordance with the teachings herein are used to optimize the in vitro culture conditions for differentiating the cells. High-throughput screens can be established to assess the effects of media components, exogenous growth factors, and attachment substrates. These substrates include viable cell feeder layers, cell extracts, defined extracellular matrix components, substrates which promote three-dimensional growth such as methylcellulose and collagen, novel cell attachment molecules, and/or matrices with growth factors or other signaling molecules embedded within them. This last approach may provide the spatial organization required for replication of complex organ architecture (as reviewed in Saltzman, Nature Medicine 4:272-273, 1998).

### EXAMPLES

The following examples are intended to illustrate but not limit the invention. While they are typical of those that might be used, other procedures known to those skilled in the art may alternatively be used.

### Example 1

### Material and Methods

Generation and Expansion of fibroblast-like cells. Frozen UCB mononuclear cells were purchased from Cambrex (2C-150A, lot: O41113, O50737, HO40926, HO41135, HO41708, HO50567, HO51251, HO51254). Generation and expansion of fibroblast-like cells were following the protocol described by Kögler (Kögler et al. 2004). Briefly, UCB mononuclear cells were cultured in low glucose DMEM+GlutaMAX™ (Invitrogen) supplemented with 30% FCS, 10⁻⁷ M dexamethasone (Sigma), 100 U/ml penicillin and 0.1 mg/ml streptomycin. Cells were initially plated at a density of 5×10⁶ cell/ml in T75 flasks and were placed in a humidified atmosphere at 37° C. and 5% CO₂. Expansion of the cells was performed in the same medium but with 5×10⁻⁸M dexamethasone. Cells were split after reaching confluence by disaggregation with 0.05% trypsin/EDTA and replating at a 1:3 expansion.

Flow cytometric analysis. Fibroblast-like cells at passage 5 were dissociated with trypsin, blocked with 10% γ-globulins, and stained with monoclonal antibody: FITC conjugated CD50, isotype control IgG2b. PE conjugated CD31, CD34, CD44, CD71, CD90, CD106 and isotype controls IgG1 and IgG2a (all antibodies from Becton Dickinson. Fluorescence activated cell sorting was carried out on a FACSCalibur instrument using the CellQuest software (BD Bioscience). At least 10,000 events were acquired for each sample, and 7-Amino-actinomycin D (7AAD) staining was used to distinguish live from dead cells.

Human ES cell culture and differentiation. The H1 and H9 HES cells were purchased from the WiCell Research Institute and initially culture on PMEFs as instructed by the provider. The cells were cultured at 37° C./5% CO₂ and 95% humidity, either on gamma irradiated (35Gy) fibroblast-like cells (˜5×10⁴ cells cm²) plated on gelatin-coated tissue culture plates or directly on on Matrigel™-coated plates. Culture media used for HES cells growing on Matrigel-coated plates was conditioned medium (CM) derived from fibroblast-like cells. Cells were passaged every 4-5 days and digested with collagenase IV (1 mg/ml, Sigma). The HES cell culture medium consisted of 80% (v/v) knockout (KO) DMEM, 10% (v/v) KO serum replacement, 2 mM L-glutamine, 10 mM nonessential amino acids (all from Invitrogen), 10% (v/v) human Plasmanate™ (Bayer Corporation), 50 μM β-mercaptoethanol (Sigma), 12 ng/ml human LIF (R&D Systems) and 10 ng/ml human bFGF (R&D Sytems). For fibroblast-like cell CM, fresh HES cell media was added into the dishes with fibroblast-like cells (˜5×10⁴ cells cm²) and CM was collected after 24 h. CM was filtered (0.22 m filter) and supplemented with 8 ng/ml human bFGF (R&D Systems) prior to use. Both HES cell lines were at passage 30 and karyotypically normal at the beginning of fibroblast-like cell culture experiments. Cell number was determined by using the Nucleocounter (New Brunswick) automated cell counting device. Population doubling time was calculated as 3.3×(log₁₀ final cell number-log₁₀ starting cell number)/hours between passage.

Immunocytochemistry. HES cells cultured on fibroblast-like cell feeder layers were fixed in 4% paraformaldehyde in PBS, permeabilized with 0.2% Trixon X-100 in PBS and blocked with 5% normal goat serum. Fixed cells were incubated with primary antibodies: OCT-3/4 (BD), SSEA-4, TRA-1-60 and TRA-1-81 (Chemicon). Goat anti-mouse IgG conjugated to Alexa 488 or 594 (Molecular probes) were used as a secondary antibodies.

Embryoid body formation. To generate embryoid bodies (EBs) from HES cells, dispase (0.2 mg-1 mg/ml) was used to lift HES cell colonies from fibroblast-like cell feeder layers. The colonies were then washed and resuspended in the HES cell medium in the absence of bFGF, human plasmanate and KO serum replacement but supplemented with 20% FBS (Hyclone), then cultured in ultra-low attachment plates (Corning). A 50% percent media replacement was carried out every 3-5 days. Cystic EBs emerged after 4 to 5 days in the culture.

Karyotype Analysis of HES cells. H9 and H1 HES cells cultured on fibroblast-like cells for 20 or more passages were used for karyotyping. Cell division was blocked by 0.25 ug/ml colcemid (Invitrogen) in metaphase at 37° C. for 1 hour. Cell were then trypsinized and re-suspended in pre-warmed hypotonic KCL (0.075M KCL) solution, incubated for 20 min at 37° C., and fixed with 3:1 methanol:acetic acid. Chromosomes were visualized using G-band staining. 20 metaphases of each sample were examined. Karyotyping analysis was performed by Georgia Esoteric Molecular Lab, Medical College of Georgia.

Teratoma Formation. Severe combined immunodeficient/beige (SCID/beige) mice were supplied by Taconic Farms Inc. USA. 1−2×10⁶ HES cells cultured on fibroblast-like cells for 20 passages were injected into the hind leg. Three mice were injected for teratoma formation per HES cell line. Equal numbers of Irradiated fibroblast-like cells were also injected separately. Teratomas were recovered after 8 weeks. Tumors were fixed overnight in 4% paraformaldehyde and processed for paraffin embedding. Histological examination was carried out after staining with hematoxylin and eosin. Animal care and the teratoma formation protocols were approved by the Johns Hopkins University School of Medicine Animal Care and Use Committee.

### Results

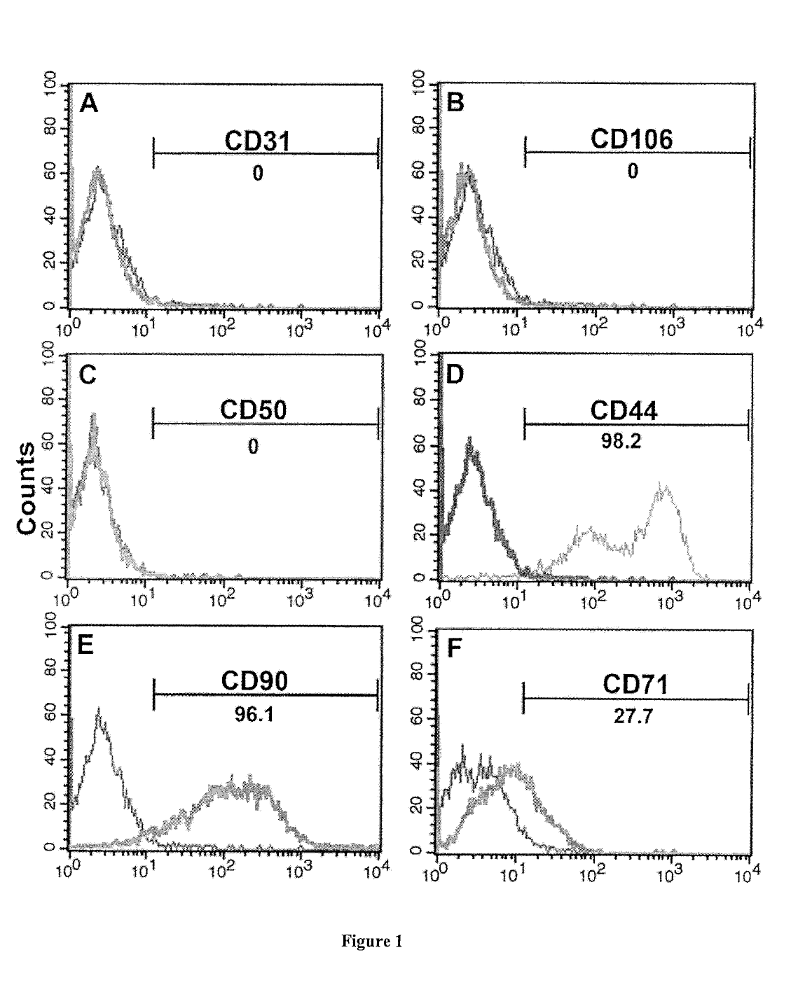

Five independent fibroblast-like cell colonies were generated from 8 lots of umbilical cord blood derived mononuclear cells (˜1×10⁸ cells per lot). The generation of fibroblast-like cell cultures was genotype dependent. From the 8 lots of umbilical cord blood derived stem cells, each of Lot HO41708 and HO51251 generated 2 fibroblast-like cell lines and Lot HO41708 generated I line, while other 5 lots generated none. Adherent cells had a spindle/fibroblast morphology similar to USSC cells described by Kögler et al. (Kögler et al. 2004; Kögler et al. 2005) but could be cultured for 6-11 passages in vitro, which is less than that of the reported USSC cells (>20 passages). Fibroblast-like cells had a similar immunophenotype to USSC. Both cell cultures are CD31, CD34, CD50, CD106 negative and positive for CD44, CD71, CD90 (Table 1 below and FIG. 1).

TABLE 1

| | Expansion | | | | | | | |
|---|---|---|---|---|---|---|---|---|
| Cell | Potential | Immune Phenotype | | | | | | |
| line | (passages) | CD31 | CD34 | CD44 | CD50 | CD71 | CD90 | CD106 |
| FLC-1 | 11 | 0 | 0 | 98.2 | 0 | 27.7 | 96.1 | 0 |
| FLC-2 | 6 | 1.1 | 0.4 | 96.3 | 0 | 28.0 | 96.6 | 5.5 |
| FLC-3 | 8 | 0.6 | 0.9 | 96.5 | 0 | 19.9 | 96.8 | 12.5 |
| FLC-4 | 10 | 2.5 | 1.8 | 96.9 | 3.9 | 21.3 | 96.4 | 6.2 |
| FLC-5 | 10 | 0 | 0 | 96.9 | 0 | 6.3 | 87.5 | 0 |

These characteristics are different from most umbilical cord blood-derived mesenchymal cell lines which are either CD90 negative (Lee et al. 2004) or CD106 positive (Bieback et al. 2004; Tisato et al. 2007). The fibroblast-like cells embodied herein were similar to HES cell-derived fibroblasts (HES-df) and human foreskin fibroblast cells (HFF), in terms of cell morphology and cell surface markers, as described by Stojkovic (Stojkovic et al. 2005) In that they all expressed cell surface markers CD44 and CD90 but lack endothelial-specific cell marker CD31 and mesenchymal cell specific marker CD106.

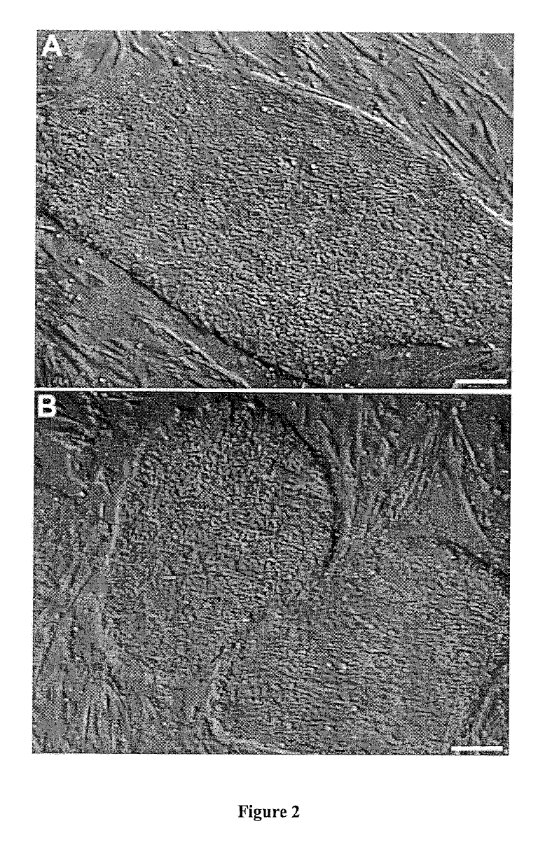

The potential of fibroblast-like cells to support HES cell growth and self-renewal using H1 and H9 cell lines was evaluated. On fibroblast-like cells line 1 (FLC-1) H1 and H9 cell lines grew with a slower population doubling time than on PMEF. On FLC-1, HES cell doubled every 45-60 hrs while on PMEF they doubled every 36-40 hrs. Both fresh and cryopreserved HES cells grew on fresh or cryopreserved FLC-1 cells. Similar to HES cell grown on HES-df but different from HES cell grown on PMEF, HES cells colonies on FLC-1 and the other FLC cultures do not grown between and under feeder cells but instead, sit on top of the feeder layer. Colonies on FLC feeder layers appeared thinner and larger as compared with the HES cells colonies on PMEFs. Because of this, HES cells cultured on FLC feeder layers were relatively easier to lift off the feeder layer following collagenase IV digestion as compared to HES cell growing on PMEF. On FLC feeder layers, HES cells within the colonies remained tightly clustered and maintained typical HES cell morphology. On FLC feeder layers, HES cell colonies grew to more than 0.2 cm in diameter without any sign of differentiation (FIG. 2A,B). This was not the case with HES cell on PMEF, which routinely differentiate as they become large colonies (data not shown).

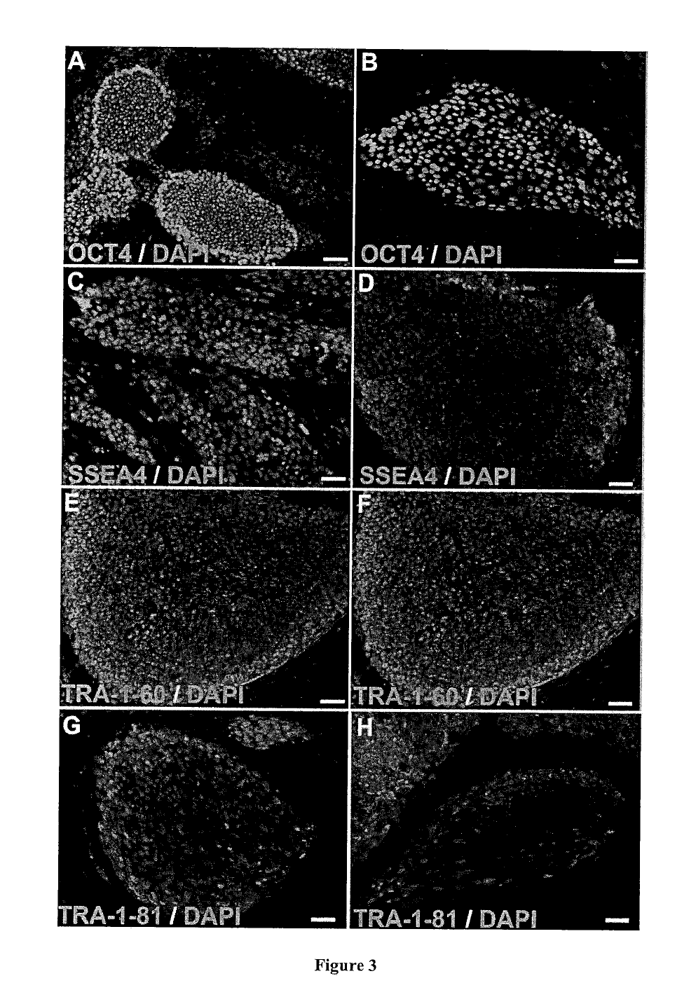

Undifferentiated status of HES cell can be characterized by the expression of a series of markers such as the transcription factor OCT-4, and the surface markers SSEA-4, Tra-1-60 and Tra-1-81. The expression of these markers on HES cell cultured on FLC-1 feeder was analyzed using immunocytochemistry. Like HES cells grown on PMEF, HES cells on FLC-1 feeder expressed typical stem cell markers of OCT-4, SSEA-4, TRA-1-60 and TRA-1-81 (FIG. 3).

FLC-1 conditioned medium (CM) was also able to support HES cells on Matrigel. H1 and H9 HES cells maintained in these conditions for 5 passages continued to display undifferentiated HES cell morphology and stem cell surface markers: 93% SSEA-4, 87% Tra-1-60 for H1 cells and 98% SSEA-4, 98% Tra-1-60 for H9 cells. Interestingly, very few cells differentiated from HES cell were observed surrounding the undifferentiated HES cells colonies as compared to HES cell cultured in the presence of PMEF CM (data not shown).

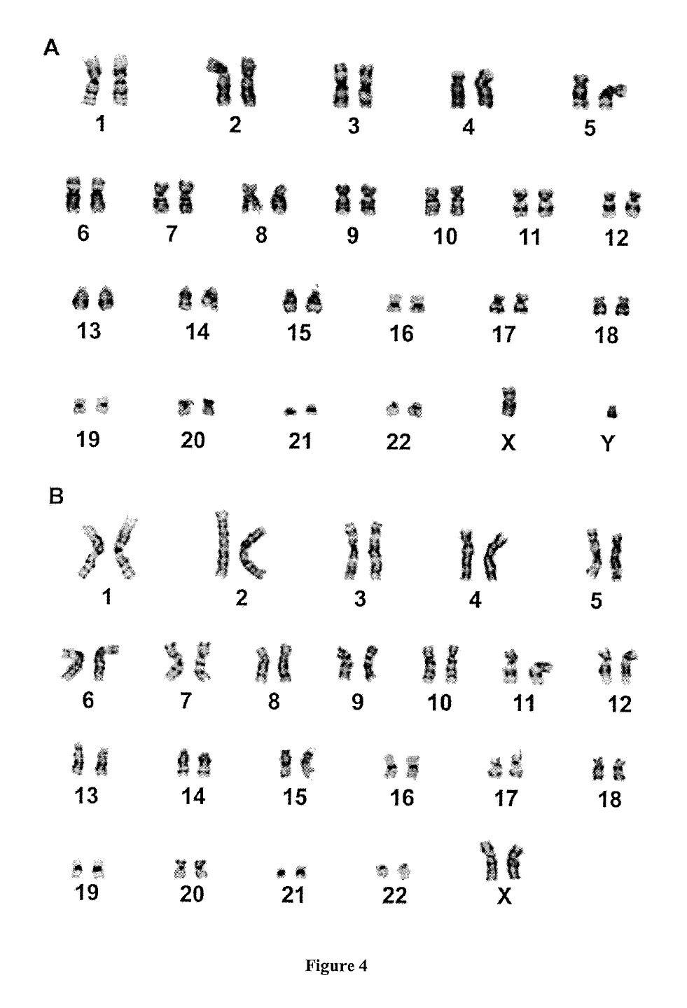

To test whether HES cells cultured on FLC-1 feeder layer maintain the genetic integrity, H1 and H9 cells after 20 passages cultured on FLC-1 feeder were karyotyped. In each case, the karyotype was normal, indicating that HES cells cultured on FLC-1 maintain their genomic integrity to the level of resolution allowed by the karyotyping technique (FIG. 4).

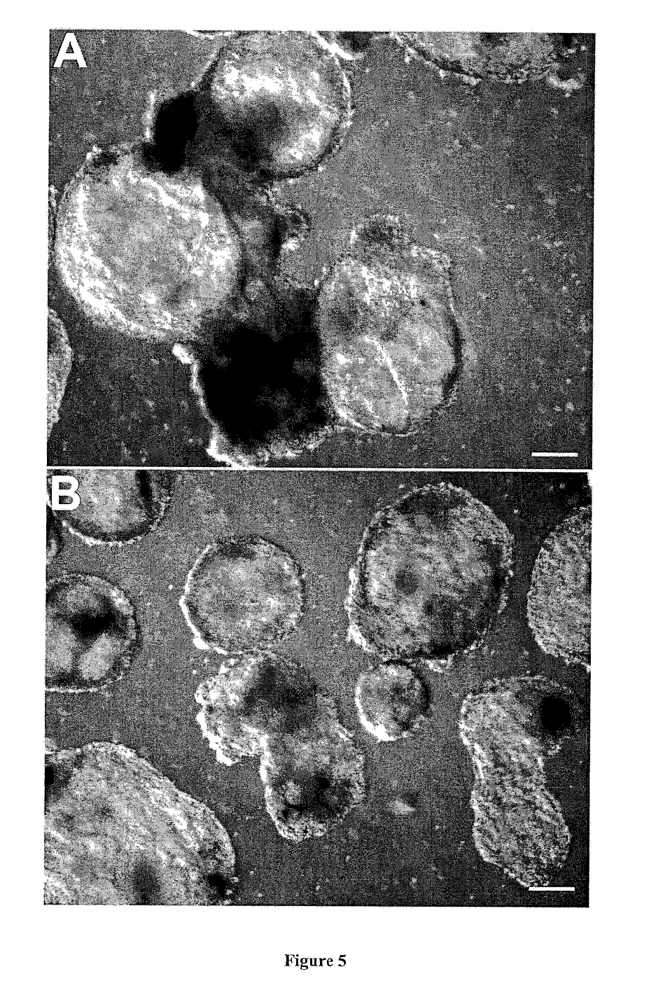

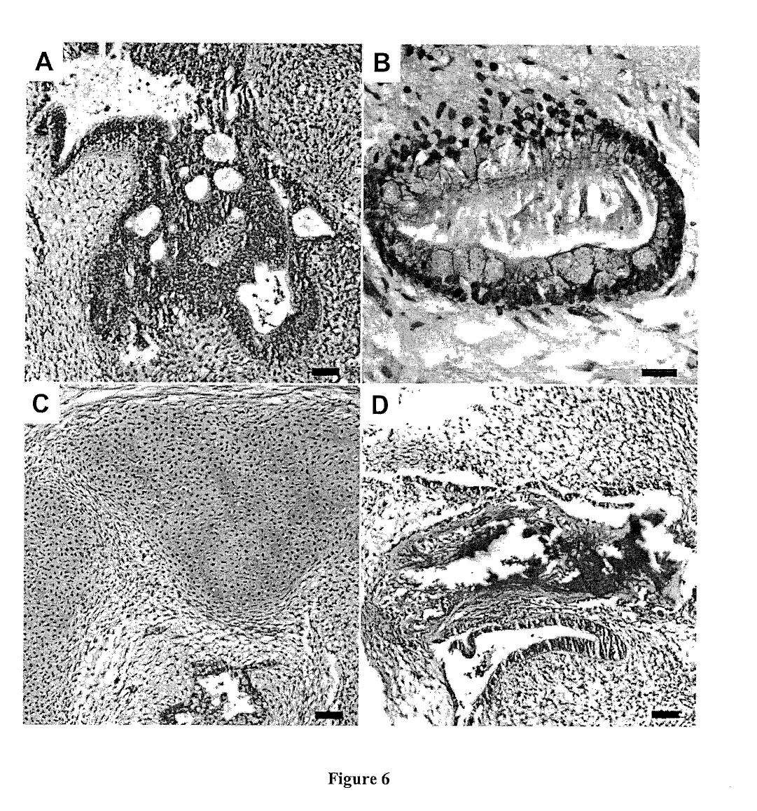

To confirm that HES cells cultured on FLC-1 feeder layer maintain their pluripotency in vitro, embryoid body formation (EB) experiments were performed. HES cells after culture on FLC-1 feeder for 20 passages were used. They formed cystic EBs in suspension with high efficiency in both cell lines (FIG. 5). To examine the in vivo pluripotency of HES cells grown on FLC-1, H1 and H9 cells after 20 passages on FLC-1 were injected into the hind leg of SCID/beige mice. Teratomas were formed in 8 weeks. The teratomas contained multiple cell types from each of the major cell lineages (FIG. 6). Thus HES cells cultured on FLC-1 for >20 passages maintain their pluripotency both in vitro and in vivo.

Four other fibroblast-like cell lines were tested (Table 1 above; FLC-2,3,4,5) for their potential to support HES cell growth and self-renewal using the H9 cell line. H9 cells grew well on these additional fibroblast-like cell lines. After 6 passages, virtually all HES cell colonies contained cells expressing OCT-4, similar to HES cell growing on FLC-1 (FIG. 3).

### Example 2

HuES-2 were observed growing on Matrigel in the presence of umbilical cord blood derived stem cell conditioned media. This conditioned media was prepared by plating umbilical cord blood derived stem cells at 1 million cells per 10 cm plate into 12 mls of HPES media. After 24 his media was harvested and sterile filtered. Prior to use on huES-2 cells, 8 ng/ml FGF2 was added to the conditioned media. The initial observation is that umbilical cord blood derived stem cell conditioned media can support the growth of HuES-e cells growth on Matrigel, but at a reduced cell proliferation rate. This is commonly observed when adapting cells to new environments. Importantly, HuES-2 cells grown in the presence of umbilical cord blood derived stem cell conditioned media had reduced spontaneous differentiation as compared to MEF conditioned media, to the point that there were no observable differentiated cells.

### REFERENCES

Amit, M., V. Margulets, H. Segev, K. Shariki, I. Laevsky, R. Coleman and J. Itskovitz-Eldor (2003). “Human feeder layers for human embryonic stem cells.” Biol Reprod 68(6): 2150-6.
Amit, M., C. Shariki, V. Margulets and J. Itskovitz-Eldor (2004). “Feeder layer- and serum-free culture of human embryonic stem cells.” Biol Reprod 70(3): 837-45.
Baker, D. E., N. J. Harrison, E. Maltby, K. Smith, H. D. Moore, P. J. Shaw, P. R. Heath, H. Holden and P. W. Andrews (2007). “Adaptation to culture of human embryonic stem cells and oncogenesis in vivo.” Nat Biotechnol 25(2): 207-15.
Beattie, G. M., A. D. Lopez, N. Bucay, A. Hinton, M. T. Firpo, C. C. King and A. Hayek (2005). “Activin A maintains pluripotency of human embryonic stem cells in the absence of feeder layers.” Stem Cells 23(4): 489-95.
Bieback, K., S. Kern, H. Kluter and H. Eichler (2004). “Critical parameters for the isolation of mesenchymal stem cells from umbilical cord blood.” Stem Cells 22(4): 625-34.
Brimble, S, N., X. Zeng, D. A. Weiler, Y. Luo, Y. Liu, I. G. Lyons, W. J. Freed, A. J. Robins, M. S. Rao and T. C. Schulz (2004). “Karyotypic stability, genotyping, differentiation, feeder-free maintenance, and gene expression sampling in three human embryonic stem cell lines derived prior to Aug. 9, 2001.” Stem Cells Dev 13(6): 585-97.
Buzzard, J. J., N. M. Gough, J. M. Crook and A. Colman (2004). “Karyotype of human ES cells during extended culture.” Nat Biotechnol 22(4): 381-2; author reply 382.
Cheng, L., H. Hammond, Z. Ye, X. Than and G. Dravid (2003). “Human adult marrow cells support prolonged expansion of human embryonic stem cells in culture.” Stem Cells 21(2): 131-42.
Cowan, C. A. et. al, Derivation of Embryonic Stem-cell Lines from Human Blastocysts. New England Journal of Medicine. 350; 13. (2004).
Draper, J. S., K. Smith, P. Gokhale, H. D. Moore, E. Maltby, J. Johnson, L. Meisner, T. P. Zwaka, J. A. Thomson and P. W. Andrews (2004). “Recurrent gain of chromosomes 17q and 12 in cultured human embryonic stem cells.” Nat Biotechnol 22(1): 53-4.
Klimanskaya, I., Y. Chung, L. Meisner, J. Johnson, M. D. West and R. Lanza (2005). “Human embryonic stem cells derived without feeder cells.” Lancet 365(9471): 1636-41.
Kogler, G., T. F. Radke, A. Lefort, S. Sensken, J. Fischer, R. V. Sorg and P. Wernet (2005). “Cytokine production and hematopoiesis supporting activity of cord blood-derived unrestricted somatic stem cells.” Exp Hematol 33(5): 573-83.
Kogler, G., S. Sensken, J. A. Airey, T. Trapp, M. Muschen, N. Feldhahn, S. Liedtke, R. V. Sorg, J. Fischer, C. Rosenbaum, S. Greschat, A. Knipper, J. Bender, O. Degistirici, J. Gao, A. I. Caplan, E. J. Colletti, G. Almeida-Porada, H. W. Muller, E. Zanjani and P. Wernet (2004). “A new human somatic stem cell from placental cord blood with intrinsic pluripotent differentiation potential.” J Exp Med 200(2): 123-35.
Lee, O. K., T. K. Kuo, W. M. Chen, K. D. Lee, S. L. Hsieh and T. H. Chen (2004). “Isolation of multipotent mesenchymal stem cells from umbilical cord blood.” Blood 103(5): 1669-75.
Ludwig, T. E., M. E. Levenstein, J. M. Jones, W. T. Berggren, E. R. Mitchell, J. L. Frane, L. J. Crandall, C. A. Daigh, K. R. Conard, M. S. Piekarczyk, R. A. Lianas and J. A. Thomson (2006). “Derivation of human embryonic stem cells in defined conditions.” Nat Biotechnol 24(2): 185-7.
Mannello, F. and G. A. Tonti (2007). “Concise review: no breakthroughs for human mesenchymal and embryonic stem cell culture: conditioned medium, feeder layer, or feeder-free; medium with fetal calf serum, human serum, or enriched plasma; serum-free, serum replacement nonconditioned medium, or ad hoc formula? All that glitters is not gold!” Stem Cells 25(7): 1603-9.
Mitalipova, M. M., R. R. Rao, D. M. Hoyer, J. A. Johnson, L. F. Meisner, K. L. Jones, S. Dalton and S. L. Stice (2005). “Preserving the genetic integrity of human embryonic stem cells.” Nat Biotechnol 23(1): 19-20.
Rajala, K., H. Hakala, S. Panula, S. Aivio, H. Pihlajamaki, R. Suuronen, O. Hovatta and H. Skottuian (2007). “Testing of nine different xeno-free culture media for human embryonic stem cell cultures.” Hum Reprod 22(5): 1231-8.
Richards, M., C. Y. Fong, W. K. Chan, P. C. Wong and A. Bongso (2002). “Human feeders support prolonged undifferentiated growth of human inner cell masses and embryonic stem cells.” Nat Biotechnol 20(9): 933-6.
Richards, M., S. Tan, C. Y. Fong, A. Biswas, W. K. Chan and A. Bongso (2003). “Comparative evaluation of various human feeders for prolonged undifferentiated growth of human embryonic stem cells.” Stem Cells 21(5): 546-56.
Shamblott, M., J. Axelman, J. Littlefield, P. Blumenthal, G. Huggins, 7. Cui, L. Cheng, and J. Gearhart, (2001) Human embryonic germ cell derivatives express a broad range of developmentally distinct markers and proliferate extensively in vitro. Proc Natl Acad Sci USA. 98: p. 113-118.
Shamblott, M. J., J. Axelman, S. Wang, E. M. Bugg, J. W. Littlefield, P. J. Donovan, P. D. Blumenthal, G. R. Huggins, and J. D. Gearhart, (1998) Derivation of pluripotent stem cells from cultured human primordial germ cells. Proc Natl Acad Sci USA. 95: p. 13726-31.
Stojkovic, P., M. Lako, R. Stewart, S. Przyborski, L. Armstrong, J. Evans, A. Murdoch, T. Strachan and M. Stojkovic (2005). “An autogeneic feeder cell system that efficiently supports growth of undifferentiated human embryonic stem cells.” Stem Cells 23(3): 306-14.
Terai, M., T. Uyama, T. Sugiki, X. K. Li, A. Umezawa and T. Kiyono (2005). “Immortalization of human fetal cells: the life span of umbilical cord blood-derived cells can be prolonged without manipulating p161NK4a/RB braking pathway.” Mol Biol Cell 16(3): 1491-9.
Thompson J A et al., 1998 Embryonic stem cell line derived from human blastocysts. Science. 282, 1145-1147.
Tisato, V., K. Naresh, J. Girdlestone, C. Navarrete and F. Dazzi (2007). “Mesenchymal stem cells of cord blood origin are effective at preventing but not treating graft-versus-host disease.” Leukemia 21(9): 1992-9.
Xu, C., M. S. Inokuma, J. Denham, K. Golds, P. Kundu, J. D. Gold and M. K. Carpenter (2001). “Feeder-free growth of undifferentiated human embryonic stem cells.” Nat Biotechnol 19(10): 971-4.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for cultivating human embryonic stem (ES) cells and maintaining the pluripotency thereof comprising growing the human embryonic stem (ES) cells in a culture medium comprising a feeder layer of umbilical cord blood derived stem cells, medium comprising secreted proteins from umbilical cord derived stem cells, or the combination thereof, wherein the umbilical cord blood derived stem cells are CD31^{neg}, CD50^{neg}, and CD71^{pos}.

2. The method of claim 1 wherein the umbilical cord blood derived stem cells are adherent, CD45^{neg}, HLA class II^{neg} stem cells.

3. The method of claim 2 wherein said adherent, CD45^{neg}, HLA class II^{neg} stem cells are CD34^{neg}, CD106^{neg}, CD44^{pos} and CD90^{pos}.

4. The method of claim 1 wherein the umbilical cord blood derived stem cells are CD31, CD34, CD50, CD106 negative, and positive for CD44, CD71, CD90.

5. The method of claim 1 wherein the feeder layer of umbilical cord blood derived stem cells are treated to halt cell division.

6. The method of claim 1 further comprising a substrate.

7. The method of claim 6 wherein the substrate is collagen I, collagen IV, fibronectin, superfibronectin, laminin, heparan sulfate proteoglycan, entactin, or any combination thereof.

8. The method of claim 7 wherein the collagen I is human type 1 collagen.

9. The method of claim 6 wherein the substrate comprises a synthetic or biosynthetic cell adhesion molecule or a mixture thereof.

10. The method of claim 1 further comprising an extracellular matrix.

11. The method of claim 10 wherein the extracellular matrix is obtained from human embryonic germ cell derivatives, human umbilical cord blood stem cells, human mesenchymal stem cells, or human fibroblasts.

12. The method of claim 11 wherein the umbilical cord blood derived stem cells are adherent, CD45^{neg}, HLA class II^{neg} stem cells.

13. The method of claim 12 wherein said adherent, CD45^{neg}, HLA class II^{neg} stem cells are CD34^{neg}, CD106^{neg}, CD44^{pos} and CD90^{pos}.

14. The method of claim 12 wherein the umbilical cord blood derived stem cells are CD31, CD34, CD50, CD106 negative, and positive for CD44, CD71, CD90.

15. The method of claim 10 wherein the extracellular matrix is EHS mouse sarcoma basement membrane.

16. A composition for cultivating human embryonic stem (ES) cells and maintaining the pluripotency thereof comprising a feeder layer of human umbilical cord blood derived stem cells, secreted proteins from human umbilical cord blood stem cells, or the combination thereof, wherein the umbilical cord blood derived stem cells are CD31^{neg}, CD50^{neg}, and CD71^{pos}.

17. The composition of claim 16 wherein the umbilical cord blood derived stem cells are adherent, CD45^{neg}, HLA class II^{neg} stem cells.

18. The composition of claim 17 wherein said adherent, CD45^{neg}, HLA class II^{neg} stem cells are CD34^{neg}, CD106^{neg}, CD44^{pos} and CD90^{pos}.

19. The composition of claim 16 wherein the umbilical cord blood derived stem cells are CD31, CD34, CD50, CD106 negative, and positive for CD44, CD71, CD90.

20. The composition of claim 16 wherein the umbilical cord blood stem cells are treated to halt cell division.

21. The composition of claim 16 further comprising a substrate.

22. The composition of claim 21 wherein the substrate is collagen I, collagen IV, fibronectin, superfibronectin, laminin, heparan sulfate proteoglycan, entactin, or any combination thereof.

23. The composition of claim 22 wherein the collagen I is human type I collagen.

24. The composition of claim 21 wherein the substrate comprises a synthetic or biosynthetic cell adhesion molecule or a mixture thereof.

25. The composition of claim 21 wherein the substrate is extracellular matrix.

26. The composition of claim 25 wherein the extracellular matrix is obtained from human embryonic germ cell derivatives, human mesenchymal stem cells, human umbilical cord derived stem cells or human fibroblasts.

27. The composition of claim 26 wherein the human umbilical cord blood derived stem cells are adherent, CD45^{neg}, HLA class II^{neg} stem cells.

28. The composition of claim 27 wherein said adherent, CD45^{neg}, HLA class II^{neg} stem cells are CD34^{neg}, CD106^{neg}, CD44^{pos} and CD90^{pos}.

29. The composition of claim 27 wherein the umbilical cord blood derived stem cells are CD31, CD34, CD50, CD106 negative, and positive for CD44, CD71, CD90.

30. The composition of claim 25 wherein the extracellular matrix is EHS mouse sarcoma basement membrane.

31. A kit for cultivating human embryonic stem (ES) cells and maintaining the pluripotency thereof, the kit comprising a first container of secreted proteins from human umbilical cord derived stem cells, a second container of substrate, and instructions for the use thereof, wherein the umbilical cord blood derived stem cells are CD31^{neg}, CD50^{neg}, and CD71^{pos}.

32. The kit of claim 31 wherein said human umbilical cord blood derived stem cells are adherent, CD45^{neg}, HLA class II^{neg} stem cells.

33. The kit of claim 32 wherein said adherent, CD45^{neg}, HLA class II^{neg} stem cells are CD34^{neg}, CD106^{neg}, CD44^{pos} and CD90^{pos}.

34. The kit of claim 31 wherein the umbilical cord blood derived stem cells are CD31, CD34, CD50, CD106 negative, and positive for CD44, CD71, CD90.

35. The kit of claim 31 wherein the substrate is collagen I, collagen IV, fibronectin, superfibronectin, laminin, heparan sulfate proteoglycan, entactin, or any combination thereof.

36. The kit of claim 35 wherein the collagen I is human type I collagen.

37. The kit of claim 31 wherein the substrate comprises a synthetic or biosynthetic cell adhesion molecule or a mixture thereof.

38. The kit of claim 31 wherein the substrate is extracellular matrix.

39. The kit of claim 38 wherein the extracellular matrix is obtained from human embryonic germ cell derivatives, human mesenchymal stem cells, human umbilical cord blood derived stem cells or human fibroblasts.

40. The kit of claim 39 wherein the human umbilical cord blood derived stem cells are adherent, CD45^{neg}, HLA class II^{neg} stem cells.

41. The kit of claim 40 wherein said adherent, CD45^{neg}, HLA class II^{neg} stem cells are CD34^{neg}, CD106^{neg}, CD44^{pos} and CD90^{pos}.

42. The kit of claim 40 wherein the umbilical cord blood derived stem cells are CD31, CD34, CD50, CD106 negative, and positive for CD44, CD71, CD90.

43. The kit of claim 38 wherein the extracellular matrix is EHS mouse sarcoma basement membrane.

44. A composition comprising pluripotent human embryonic stem (ES) cells and secreted proteins from human umbilical cord blood derived stem cells, in combination with a substrate, wherein the umbilical cord blood derived stem cells are CD31^{neg}, CD50^{neg}, and CD71^{pos}.

45. The composition of claim 44 wherein the umbilical cord blood derived stem cells are adherent, CD45^{neg}, HLA class II^{neg} stem cells.

46. The composition of claim 45 wherein said adherent, CD45^{neg} HLA class II^{neg} stem cells are CD34^{neg}, CD106^{neg}, CD44^{pos} and CD90^{pos}.

47. The composition of claim 44 wherein the umbilical cord blood derived stem cells are CD31, CD34, CD50, CD106 negative, and positive for CD44, CD71, CD90.

48. The composition of claim 44 wherein the substrate is collagen I, collagen IV, fibronectin, superfibronectin, laminin, heparan sulfate proteoglycan, entactin, or any combination thereof.

49. The composition of claim 48 wherein the collagen I is human type 1 collagen.

50. The composition of claim 44 wherein the substrate comprises a synthetic or biosynthetic cell adhesion molecule or a mixture thereof.

51. The composition of claim 44 wherein the substrate is extracellular matrix.

52. The composition of claim 51 wherein the extracellular matrix is obtained from human embryonic germ cell derivatives, human mesenchymal stem cells, human umbilical cord blood derived stem cells or human fibroblasts.

53. The composition of claim 52 wherein the human umbilical cord blood derived stem cells are adherent, CD45^{neg}, HLA class II^{neg} stem cells.

54. The composition of claim 53 wherein said adherent, CD45^{neg}, HLA class II^{neg} stem cells are CD34^{neg}, CD106^{neg}, CD44^{pos} and CD90^{pos}.

55. The composition of claim 53 wherein the umbilical cord blood derived stem cells are CD31, CD34, CD50, CD106 negative, and positive for CD44, CD71, CD90.

56. The composition of claim 51 wherein the extracellular matrix is EHS mouse sarcoma basement membrane or human extracellular matrix.

57. A composition comprising pluripotent human embryonic stem (ES) cells and human umbilical cord blood derived stem cells.

58. The composition of claim 57 wherein the umbilical cord blood derived stem cells are adherent, CD45^{neg}, HLA class II^{neg} stem cells.

59. The composition of claim 58 wherein said adherent, CD45^{neg}, HLA class II^{neg} stem cells are CD34^{neg}, CD106^{neg}, CD44^{pos} and CD90^{pos}.

60. The composition of claim 58 wherein the umbilical cord blood derived stem cells are CD31, CD34, CD50, CD106 negative, and positive for CD44, CD71, CD90.

61. The composition of claim 57 wherein the human umbilical cord blood derived stem cells are treated to halt cell division.

62. The composition of claim 57 further comprising a substrate.

63. The composition of claim 62 wherein the substrate is collagen I, collagen IV, fibronectin, superfibronectin, laminin, heparan sulfate proteoglycan, entactin, or any combination thereof.

64. The composition of claim 63 wherein the collagen I is human type 1 collagen.

65. The composition of claim 62 wherein the substrate comprises a synthetic or biosynthetic cell adhesion molecule or a mixture thereof.

66. The composition of claim 62 wherein the substrate is extracellular matrix.

67. The composition of claim 66 wherein the extracellular matrix is obtained from human embryonic germ cell derivatives, human mesenchymal stem cells, human umbilical cord blood derived stem cells or human fibroblasts.

68. The composition of claim 67 wherein the human umbilical cord blood derived stem cells are adherent, CD45^{neg}, HLA class II^{neg} stem cells.

69. The composition of claim 68 wherein said adherent, CD45^{neg}, HLA class II^{neg} stem cells are CD34^{neg}, CD106^{neg}, CD44^{pos} and CD90^{pos}.

70. The composition of claim 68 wherein the umbilical cord blood derived stem cells are CD31, CD34, CD50, CD106 negative, and positive for CD44, CD71, CD90.

71. The composition of claim 66 wherein the extracellular matrix is EHS mouse sarcoma basement membrane or human extracellular matrix.

72. Cultured pluripotent human embryonic stem (ES) cells obtained by the process of 1) providing a culture medium comprising a composition of claim 16; 2) introducing human embryonic stem cells thereto; and 3) growing the human embryonic stem cells therein to produce cultured pluripotent human embryonic stem cells.

73. A method for obtaining a pluripotent human embryonic cell line comprising the steps of
1) isolating cells from the inner cell mass of a pre-implantation embryo,
2) introducing the cells of (1) into a culture medium comprising the composition of claim 16,
3) growing the human embryonic stem cells over several passages in the culture medium, thereby obtaining a human embryonic cell line derived from the pre-implantation
